(19) United States
(12) Patent Application Publication
(10) Pub. No.: US 2025/0265715 A1
(43) Pub. Date: Aug. 21, 2025

(54) ADAPTATION OF PET DATA ACQUISITION PARAMETERS

(71) Applicant: Siemens Medical Solutions USA, Inc., Malvern, PA (US)

(72) Inventors: Sven Zuehlsdorff, Oak Brook, IL (US); Mario Zeiss, Barrington, IL (US); James Williams, Knoxville, TN (US)

(21) Appl. No.: 18/581,906

(22) Filed: Feb. 20, 2024

(51) Int. Cl. G06T 7/00; G06T 11/00

(52) U.S. Cl. CPC G06T 7/0014; G06T 11/003; G06T 2207/10081; G06T 2207/10104; G06T 2207/20084

(57) ABSTRACT

Systems and methods include determination of an anatomical image of an object, input of the anatomical image to a trained neural network to generate a synthetic functional image, acquisition of molecular imaging data of the object based on acquisition parameters, reconstruction of a functional image based on the molecular imaging data, determination of a difference between the functional image and the synthetic functional image, change of one of the acquisition parameters based on the difference, acquisition of second molecular imaging data of the object based on the changed acquisition parameters, and reconstruction of a second functional image based on the second molecular imaging data.

### BACKGROUND

Molecular Imaging (MI) is a well-established diagnostic imaging approach to assess the presence and severity of disease, design patient therapies, and facilitate patient monitoring. MI generates functional images which represent biological processes (e.g., glucose metabolism, receptor affinity) occurring within a patient.

A clinician inspects such functional images for characteristics associated with the clinical issues at hand. For example, in an oncological context, a clinician typically identifies areas of increased tracer uptake to assess the extent of metastasized cancer. Neurodegenerative conditions may be identified via hyper or hypo tracer uptake in brain regions. Hyper or hypo tracer uptake in the heart may be indicative of cardiovascular disease. Moreover, systemic diseases (e.g., diabetes) may manifest themselves through global and subtle changes in a functional image.

To assist inspection of a functional image, a corresponding anatomical image may be generated using Computed Tomography (CT) or Magnetic Resonance (MR) imaging. Accordingly, MI is often used in a hybrid fashion that integrates a functional imaging modality with an anatomical imaging modality (e.g. PET/CT, SPECT/CT, PET/MR).

Patient outcomes may depend on a clinician's ability to identify clinically-relevant information based on acquired images. Systems are desired to improve the detectability of clinically-relevant information within the images. One approach includes improving the quality of MI images through known mechanisms. However, standard-of-care acquisition data acquisition protocols (e.g., scan time, data gating) in MI are typically well-defined for a given clinical indication and result in acceptable image quality for most patients. Changing these data acquisition protocols to improve image quality will likely result in increased costs (e.g., due to an increase in imaging room time, processing time, processing power) and may in most cases result in little or no improvement in the identification of clinically-relevant information.

Systems are desired to improve the detectability of clinically-relevant information within MI images in a cost and time-efficient manner.

### BRIEF DESCRIPTION OF THE DRAWINGS

FIG. 1 is a block diagram of a system to adapt data acquisition parameters during data acquisition according to some embodiments.

FIG. 2 is a flow diagram of a process to adapt data acquisition parameters during data acquisition according to some embodiments.

FIGS. 3A through 3E represent PET data acquisition according to some embodiments.

FIG. 4 illustrates training of a neural network to generate a synthetic PET image based on a CT image according to some embodiments.

FIG. 5 is a flow diagram of a process to adapt data acquisition parameters according to some embodiments.

FIGS. 6A and 6B represent adaptation of PET data acquisition parameters according to some embodiments.

FIG. 7 is a block diagram of a PET-CT imaging system according to some embodiments.

### DETAILED DESCRIPTION

The following description is provided to enable any person in the art to make and use the described embodiments. Various modifications, however, will remain apparent to those in the art.

Embodiments may facilitate the generation of MI images which are well-suited to the extraction of clinically-relevant information. Briefly, “normal” tracer uptake, i.e., activity, may be initially estimated based on an acquired anatomical image of a patient. An MI image is then acquired to determine actual activity within the patient, and differences between the MI image and the “normal” activity are identified. In some embodiments, additional information (e.g., gender, expected disease, extent of disease) is used to assist in identifying the differences. Based on the nature of the differences, MI data acquisition parameters are adapted to change how data is acquired from regions associated with the differences. An MI image is reconstructed from subsequently-acquired data and may then be used to identify clinically-relevant information.

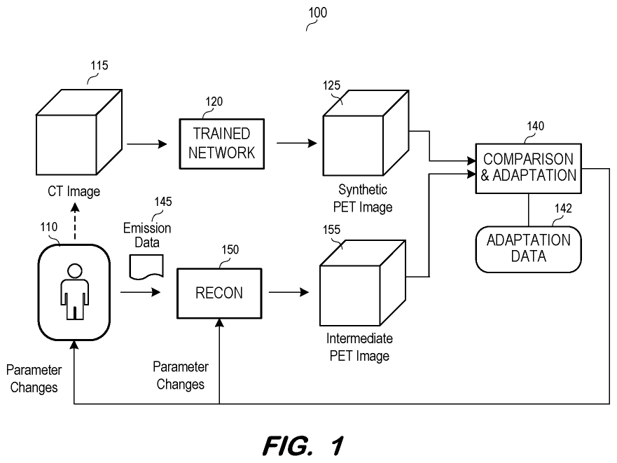

FIG. 1 is a block diagram of system 100 to adapt data acquisition parameters during data acquisition according to some embodiments. The illustrated components of system 100 may be implemented in computer hardware, in program code and/or in one or more computing systems executing such program code as is known in the art. Such a computing system may include one or more processing units which execute program code stored in a memory system. More than one functional component may be implemented by a single computing system in some embodiments. One or more of the computing systems may comprise a virtual machine, and one-or more computing systems may comprise a cloud-based compute resource providing on-demand scalability and failure recovery.

According to MI techniques, a radiotracer is administered to or ingested by a patient and radiation (e.g., gamma rays) is emitted from within the patient and captured by detectors. The detectors may capture a plurality of sets of two-dimensional emission data, or projection images, each of which may be considered a “frame” and is associated with a respective time period. The time period of two frames may overlap in some scenarios. Embodiments are not limited to projection images. Any format of raw image data may be used, including but not limited to list mode data, sinograms, and photon counting data.

The examples provided herein are directed to Positron Emission Tomography (PET) imaging, but embodiments are not limited thereto. For example, embodiments may be implemented using other MI modalities including Single Photon Computed Tomography (SPECT) imaging. Similarly, anatomical images are described herein as being acquired using CT imaging but may be acquired using MR imaging.

In this regard, scanner 110 may comprise a PET/CT scanner which generates three-dimensional CT image 115 of a patient using any suitable imaging protocol. CT image 115 is input to trained neural network 120 which generates three-dimensional synthetic PET image 125 based thereon. Network 120 has been trained as will be described below to generate a simulated PET image based on an input CT image. The simulated PET images output by network 120 are intended to represent a “normal” patient state (i.e., an expected tracer uptake and distribution as estimated from anatomical information). Network 120 may comprise hardware and software specifically-intended for executing algorithms based on a specified network architecture and trained parameters.

Scanner 110 may generate emission data 145 substantially contemporaneously with the acquisition of CT image 115. For example, a CT imaging system of scanner 110 may be operated to acquire CT image 115 while a patient lies in a given position on a bed of scanner 110, and a PET imaging system of scanner 110 may be operated shortly thereafter to acquire emission data 145 while the patient remains on the bed in the given position. Since the geometric transformation (if any) between coordinates of the CT imaging system and the PET imaging system is known, resulting images may be easily registered with one another.

Scanner 110 generates emission data 145 using any suitable PET imaging protocol. For example, a radiopharmaceutical tracer is introduced into a patient body via arterial injection. Radioactive decay of the tracer generates positrons which eventually encounter electrons and are annihilated thereby. The annihilation produces two 511keV photons which travel in approximately opposite directions. A ring of detectors surrounding the body detects 511 keV photons and identifies “coincidences” based thereon.

A coincidence is identified when two detector crystals disposed on opposite sides of the body detect the arrival of two photons within a short time window indicating that the two photons arose from the same positron annihilation. Because the two “coincident” photons travel in approximately opposite directions, the locations of the two detector crystals determine a Line-of-Response (LOR) along which an annihilation may have occurred. Time-of-flight (TOF) PET additionally measures the difference between the detection times of the two photons arising from the annihilation. This difference may be used to estimate a particular position along the LOR at which the annihilation occurred.

Emission data 145 may comprise raw (i.e., list-mode) data and/or sinograms. List-mode data may represent each detected annihilation as an LOR between two detector crystals, the time at which each photon of the annihilation reached each detector crystal and the difference between the arrival times of the two photons. A sinogram is a data array of the angle versus the displacement of each LOR. A sinogram includes one row containing the LOR for a particular azimuthal angle q. Each of these rows corresponds to a one-dimensional parallel projection of the tracer distribution at a different coordinate. A sinogram stores the location of the LOR of each coincidence such that all the LORs passing through a single point in the volume trace a sinusoid curve in the sinogram.

Reconstruction unit 150 receives emission data 145. Reconstruction unit 110 performs a reconstruction operation on emission data 145 and outputs three-dimensional intermediate PET image 155. Reconstruction unit 150 may execute any suitable reconstruction operation that is or becomes known. Reconstruction may include subtraction of random coincidences and scatter coincidences from data 145, application of attenuation correction based on a linear attenuation coefficient map (“mu-map”) derived from CT image 115, and any other suitable reconstruction steps. The reconstruction steps may be similar to reconstruction steps which were used to generate ground truth normal PET images from historical CT images for use in training model 120.

Intermediate PET image 155 is denoted as such because it is not intended to be a final PET image resulting from the inventive system. For example, emission data 145 may be acquired during a first portion of a full scan (i.e., prior to completion of the full scan) and intermediate PET image 155 may be reconstructed prior to completion of the full scan. In another example, intermediate PET image 155 may comprise a “PET scout” acquired at a higher scanning speed than a typical MI scan. The higher scanning speed may result in a lower signal-to-noise ratio within intermediate PET image 155 than would be achieved at traditional scanning speeds.

Comparison and adaptation component 140 compares synthetic PET image 125 and intermediate PET image 155 to detect differences therebetween, and to determine whether a detected difference requires adaptation of the data acquisition parameters which were used to generate intermediate PET image 155. A detected difference may comprise greater activity in a given region, lesser activity in a given region, and/or a different spatial distribution of activity. Activity within a region may be represented by image brightness, signal strength, count rate, etc.

The detection of differences may utilize thresholds. For example, a difference may be detected if the brightness of a region of intermediate PET image 155 is greater than a brightness of the region of synthetic PET image 125 by more than a first threshold. Similarly, a difference may be detected if the brightness of a region of intermediate PET image 155 is less than a brightness of the region of synthetic PET image 125 by more than a second threshold. The first and second thresholds may differ. The first and second thresholds may also be region-specific in some embodiments.

Adaptation data 142 specifies data acquisition parameter adaptations corresponding to various differences. The presence of disease typically manifests itself in hyperactivity (e.g., lesions of increased glucose metabolism, affinity to specific receptors) or hypoactivity (e.g., reduced perfusion, injured tissue). Disease may be focal (e.g., metastasis), organ specific (e.g., kidney failure) or systemic (e.g., diabetes, peripheral artery disease).

In one example, detected hypoactivity in intermediate PET image 155 with respect to the same region of synthetic PET image 125 may call for greater signal-to-noise ratio (SNR) in the region. Accordingly, adaptation data 142 may associate hypoactivity with parameter adaptations including but not limited to increasing scan time (to collect more counts), reducing table speed (to increase SNR), moving the hypoactive region to an area of highest scanner sensitivity (to increase sensitivity), and/or using a higher-sensitivity acquisition mode. Embodiments are not limited to any particular data acquisition parameter adaptations nor to any particular correspondences between detected differences and data acquisition parameter adaptations.

In another example, detected hyperactivity in a region of intermediate PET image 155 with respect to the same region of synthetic PET image 125 may call for increased resolution of the region. Adaptation data 142 may associate such hyperactivity with parameter adaptations including but not limited to reducing acceptance angle (to increase spatial resolution). If a difference in activity distribution is detected, adaptation data 142 may specify use of data-driven gating (to improve quantification to eliminate respiratory artifacts), use of point-spread function reconstruction (to improve detectability), decreasing table speed (to increase SNR), and/or to increase the scan field (to capture more pathologies). Adaptation data 142 may specify, for the same difference (e.g., a same level of hypoactivity), different acquisition parameter adaptations for different regions.

If one or more differences are detected, comparison and adaptation component 140 determines corresponding acquisition parameter adaptations from adaptation data 142 and initiates changing of the corresponding one or more parameters. The change(s) may be effected at scanner 110 and/or at reconstruction unit 150. In the latter regard, an adaptation may change a parameter or logic of a reconstruction algorithm used to generate a final PET image. An adapted data acquisition parameter may be used only for scanning the region exhibiting the difference corresponding to the data acquisition parameter or may be used for the remainder of a scan.

Comparison and adaptation component 140 may detect differences between images 155 and 125, determine corresponding acquisition parameter change(s), and initiate the changes while scanner 110 continues to acquire emission data from the patient. Accordingly, data which is subsequently acquired during the scan will be acquired according to the changed acquisition parameter(s). The scan may then complete and a final PET image may be reconstructed based on the emission data acquired after the parameter change(s) and, in some embodiments, on the emission data acquired prior to the parameter change(s).

Alternatively, in some embodiments, a second intermediate PET image is generated based on emission data acquired after the parameter change(s) and the above process repeats to compare the second intermediate PET image with synthetic PET image 125, to detect differences, to determine corresponding acquisition parameter change(s), and to initiate the changes while scanner 110 continues to acquire emission data from the patient.

In still other embodiments, intermediate PET image 155 is a PET scout and scanner 110 does not acquire emission data during identification of differences and determination of corresponding acquisition parameter changes. Rather, scanner 110 is configured based on determined acquisition parameter changes and a full scan is performed using the configuration to acquire emission data which is reconstructed into a final PET image.

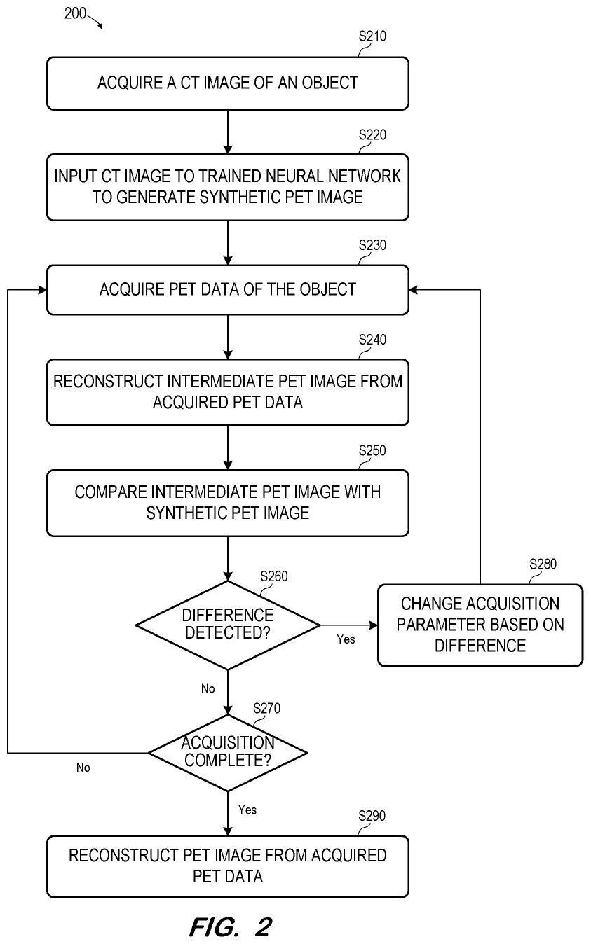

FIG. 2 is a flow diagram of process 200 to adapt data acquisition parameters during data acquisition according to some embodiments. Process 200 may be performed by any combination of hardware and software that is or becomes known. Program code embodying processes described herein may be stored by any non-transitory tangible medium, including a fixed disk, a volatile or non-volatile random-access memory, a DVD, a Flash drive, and a magnetic tape, and executed by any suitable processing unit, including but not limited to one or more microprocessors, microcontrollers, processor cores, and processor threads. Embodiments are not limited to the examples described below.

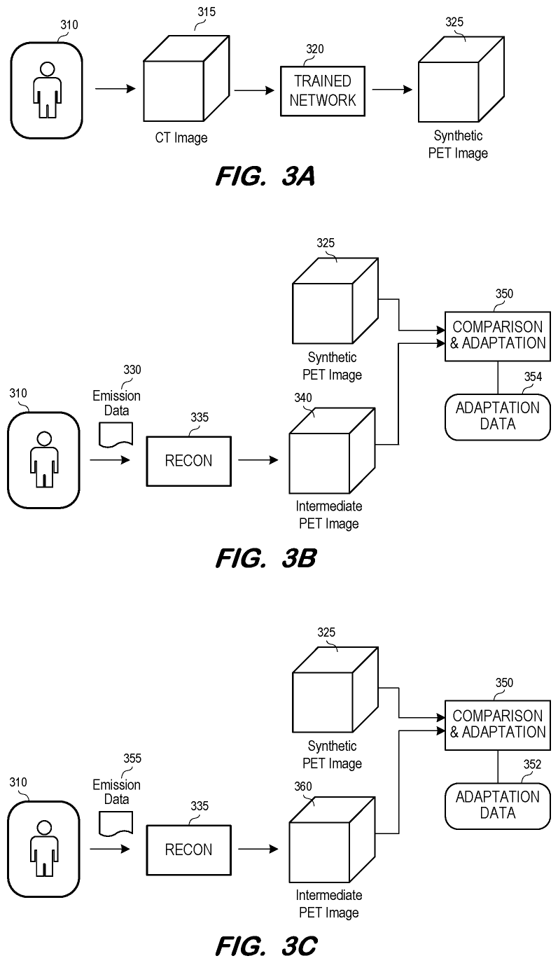

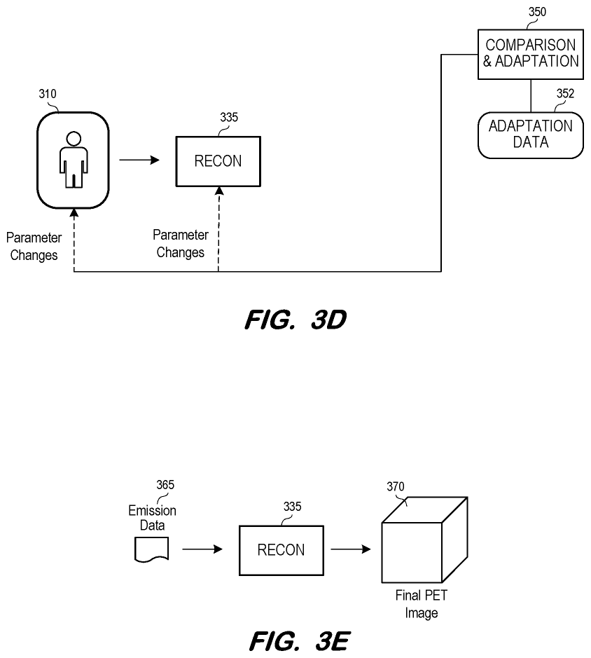

A CT image of an object is acquired at S210 using any suitable CT imaging protocol. The CT image is input to a trained neural network at S220 to generate a synthetic PET image. FIG. 3A illustrates S210 and S220 according to some embodiments. Scanner 310 executes a CT imaging protocol as is known in the art to acquire projection images of a patient and reconstruct CT image 315 from the projection images. Next, CT image 315 is input to trained neural network 320 to generate synthetic PET image 325.

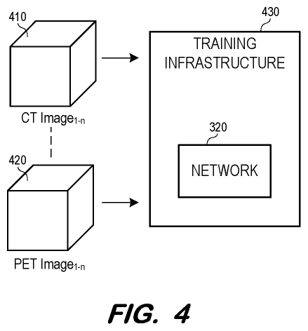

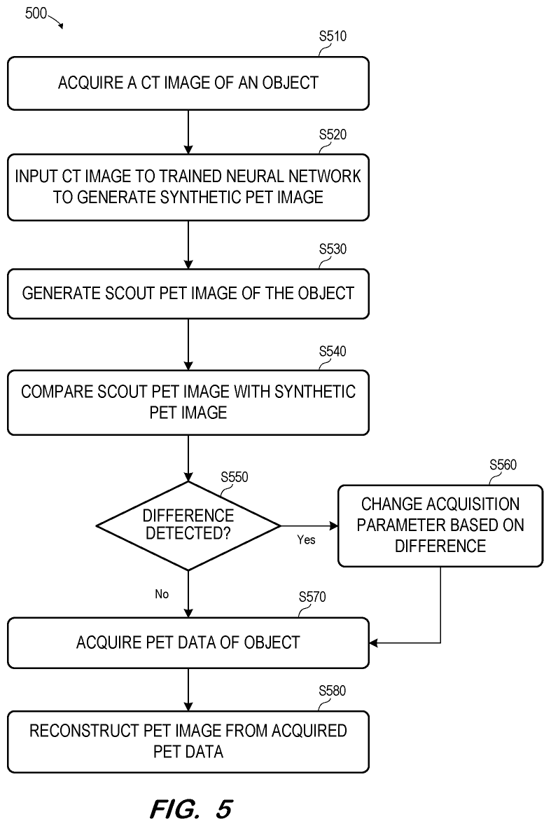

FIG. 4 illustrates training of neural network 320 to generate a synthetic PET image based on a CT image according to some embodiments. Network 320 may comprise, for example, a generator of a Generative Adversarial Network (GAN) having trainable parameters as is known in the art. Network 320 may comprise any type of supervised or unsupervised learning-compatible network, algorithm, decision tree, etc. to receive image data and to output image data that is or becomes known, including but not limited to convolutional neural networks, cycle-GAN networks and U-Net networks.

The training data of FIG. 4 consists of N CT images 410 and corresponding N PET images 420. As indicated by the dashed line, each of images 410 may have been acquired contemporaneously with a corresponding one of images 420. For example, each pair of corresponding images 410 and 420 (e.g., CT image₁ and PET image₁) may have been acquired by a PET/CT scanner, contemporaneously and of the same patient. Each of images 420 may represent normal tracer uptake and distribution in view of the anatomical information of its corresponding CT image 420.

To increase the usefulness of trained network 320, CT images 410 may be generated in a similar manner (i.e., using similar acquisition and reconstruction parameters) as those acquired at S210. Similarly, PET images 420 may be generated similarly (i.e., using similar acquisition and reconstruction parameters) as the intermediate PET images described herein.

Network 320 may comprise a plurality of layers of neurons which receive input, change internal state according to that input, and produce output depending on the input and internal state. The output of certain neurons is connected to the input of other neurons to form a directed and weighted graph. The weights as well as the functions that compute the internal states are iteratively modified during training. Thusly-trained network 320 may be implemented by a set of linear equations, executable program code, a set of hyperparameters defining a model structure and a set of corresponding weights, or any other representation of the mapping of input to output which was learned as a result of the training.

Training infrastructure 430 includes any components suitable to train network 320 based on images 410 and 420 in view of the type of network 320. For example, in the case of a GAN, network 320 may comprise a generator and training infrastructure 430 may include a discriminator, a random input generator, and a loss layer to determine generative loss and discrimination loss. After training, network 320 may be deployed as shown in FIG. 3A to generate a synthetic PET image based on an input CT image.

Returning to process 200, acquisition of PET data (i.e., emission data) of the object begins at S230. After some time, and prior to completion of a full PET scan, an intermediate PET image is reconstructed at S240 from the emission data collected thus far. Next, at S250, the intermediate PET image is compared to the synthetic PET image to detect any differences therebetween as described above.

FIG. 3B illustrates S230-S250 according to some embodiments. Scanner 310 acquires emission data 330 of a patient during a first period of a PET scan. Acquired emission data 330 is used to reconstruct intermediate PET image 340 at S240, and comparison and adaptation component 350 uses adaptation data 354 to compare intermediate PET image 340 with synthetic PET image 325 and to detect any differences therebetween at S250.

It is assumed that no difference is detected at S260 and flow therefore continues to S270. At S270, it is determined that the acquisition of data (i.e., the PET scan) is not yet complete. Flow therefore returns to S230, to continue to acquire PET data of the object. As noted above, scanner 310 may have been also acquiring emission data during the prior execution of S240-S270. In some embodiments, data acquisition pauses during some or all of S240-S270.

FIG. 3C depicts continued acquisition of PET data during a next iteration of S230, during which scanner 310 acquires new emission data 355. Reconstruction component 335 uses emission data 355 to reconstruct next intermediate PET image 360 at S240, and comparison and adaptation component 350 uses adaptation data 354 to compare intermediate PET image 360 with synthetic PET image 325 and to detect differences therebetween at S250.

Assuming that a difference is detected at S250, flow proceeds to S280 to change at least one data acquisition parameter based on the difference. FIG. 3D illustrates an example of S280, at which comparison and adaptation component 350 determines one or more acquisition parameter adaptations from adaptation data 352 based on the detected difference and initiates adaptation of the corresponding one or more parameters. The parameters may be related to the operation of scanning hardware and therefore changed at scanner 310 and/or related to reconstruction and therefore changed at reconstruction component 335 (which itself may be a component of scanner 310).

Flow may continue in this manner until it is determined at S270 that PET data acquisition is complete. According to some embodiments in which the acquisition parameters can be changed only once during a PET scan, flow proceeds directly from S280 to S270 and pauses until PET data acquisition is complete. In some embodiments, it may be determined at S280 that no acquisition parameters need to be changed based on the detected difference and flow also proceeds directly from S280 to S270. A PET image is reconstructed from the acquired PET data at S290 once it is determined at S270 that PET data acquisition is complete.

FIG. 3E illustrates reconstruction of final PET image 370 by reconstruction component 355 from emission data 365. Emission data 365 may comprise all of the emission data acquired during process 200. That is, in some embodiments, emission data 365 may comprise emission data acquired prior to a change in a data acquisition parameter and emission data acquired after the change. Emission data 365 may include emission data used to reconstruct intermediate PET images during process 200, as well as emission data which was acquired but not used to reconstruct an intermediate PET image. In this regard, each intermediate PET image may be reconstructed from all emission data which was previously acquired during the scan, from only emission data which was acquired since a last parameter adaptation, from only acquired emission data which was not used to reconstruct any prior intermediate image, or from any combination thereof.

FIG. 5 is a flow diagram of process to adapt data acquisition parameters according to some embodiments. According to process 500, a scout image is used to identify differences and determine any corresponding data acquisition parameter changes. A full scan is then performed using any determined changes.

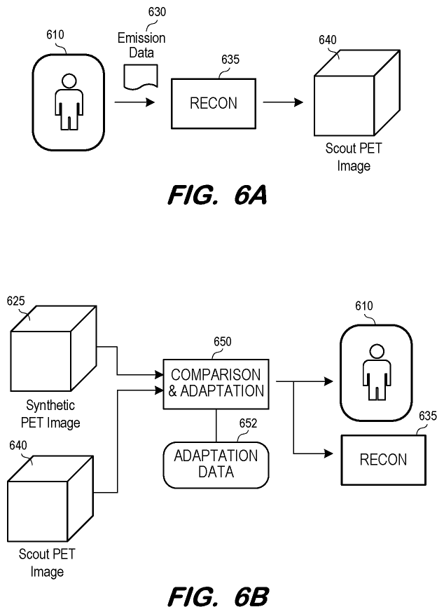

S510 and S520 may proceed as described above with respect to S210 and S220 to generate a synthetic PET image. Next, a scout image is acquired at S530. FIG. 6A illustrates operation of scanner 610 to acquire emission data 630 and to reconstruct scout PET image 640 from emission data 630.

The scout image may be acquired after tracer injection and immediately prior to a planned full scan. In some embodiments, the scout image is acquired using a faster table speed and a much shorter scan time than the forthcoming full scan. The data acquisition parameters of the scout image may be configured in view of a particular difference of concern.

The scout PET image is compared with the synthetic PET image at S540 to detect any differences between the two images. If a difference requiring a change in acquisition parameter is detected, flow proceeds from S550 to S560 to change an acquisition parameter based on the difference.

FIG. 6B depict a comparison at S540 between synthetic PET image 625 and scout PET image 640. As described above, component 650 may use adaptation data 652 to detect differences and determine any corresponding changes to data acquisition parameters. The changes may be applied to already-planned acquisition parameters of scanner 610 and/or reconstruction component 635.

For example, it may be planned to execute a full PET scan using data acquisition parameters which represent the current standard of care. However, if a difference is detected at S540, one or more of the parameters are changed at S560 prior to executing the full PET scan. The parameters of the full PET scan are changed if no difference is detected at S540. A full PET scan is executed to acquire PET data of the object at S570 and a PET image is reconstructed from the PET data at S580.

Embodiments may automatically provide improved and targeted imaging when needed, while providing images which at least meet the standard of care.

Embodiments may be particularly useful in scenarios which present substantial discrepancies between normal and actual activity. These scenarios include but are not limited to focal disease, systemic disease, and inaccurate dosing. More specifically, focal uptake patterns are typically found in primary disease or metastatic disease. Systemic conditions (such as diabetes) may lead to a global re-distribution of PET tracers that is not obvious to the clinician (e.g., decreased global metabolism). Systems conditions also include changes that impact entire organs (e.g., triple vessel disease of the heart, kidney failure). Accurate dosing may present practical challenges such as infiltration, pooling or subcutaneous injection, incomplete injection, or incorrect dosage. However, viable images can still be acquired in many cases despite inaccurate dosing, for example by increasing scan time to collect sufficient counts to reconstruct diagnostically-viable images.

Incidental findings are non-anticipated findings that are recognized during a study, and some incidental findings call for immediate treatment. For example, since cardiac images often contain portions of the liver, a cardiac exam can reveal an incidental finding in the liver. In case of incidental findings, data acquisition parameters may be adapted to capture all potential areas of interest with appropriate diagnostic image quality.

Embodiments may provide for quantification of areas of discrepancy. In particular, all potential lesions or functional changes should be detected, and data acquisition parameters should be adapted to enable accurate characterization thereof. This adaptation may include application of appropriate motion correction methods or minimal statistics to quantify the signal.

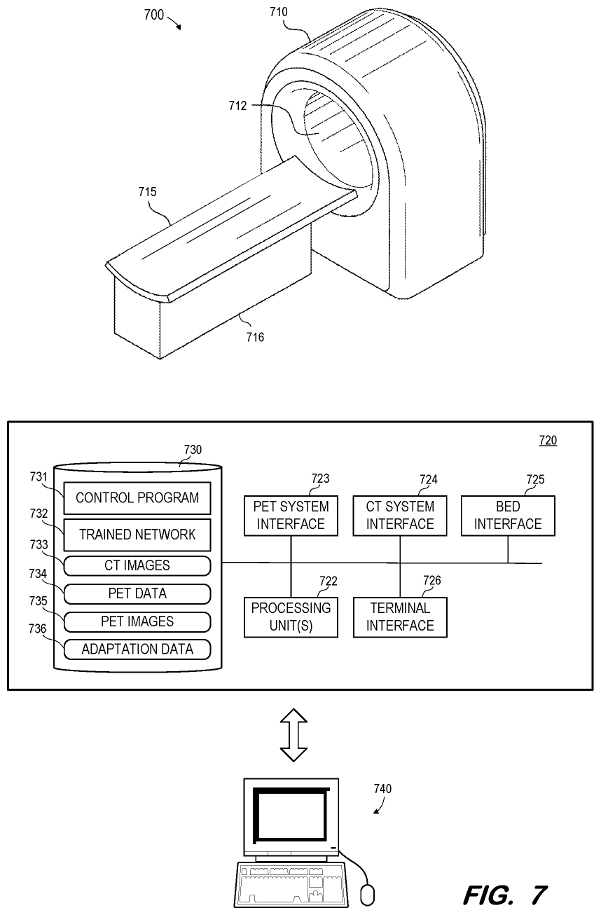

FIG. 7 illustrates PET/CT scanner 700 to execute one or more of the processes described herein. Embodiments are not limited to scanner 700 or to a multi-modality imaging system.

Scanner 700 includes gantry 710 defining bore 712. As is known in the art, gantry 710 houses PET imaging components for acquiring PET image data and CT imaging components for acquiring CT image data. The CT imaging components may include one or more x-ray tubes and one or more corresponding x-ray detectors as is known in the art. The PET imaging components may include any number or type of detectors including background radiation-emitting crystals and disposed in any configuration as is known in the art.

Bed 715 and base 716 are operable to move a patient lying on bed 715 into and out of bore 712 before, during and after imaging. In some embodiments, bed 715 is configured to translate over base 716 and, in other embodiments, base 716 is movable along with or alternatively from bed 715.

Movement of a patient into and out of bore 712 may allow scanning of the patient using the CT imaging elements and the PET imaging elements of gantry 710. Bed 715 and base 716 may provide continuous bed motion and/or step-and-shoot motion during such scanning according to some embodiments.

Control system 720 may comprise any general-purpose or dedicated computing system. Accordingly, control system 720 includes one or more processing units 722 configured to execute processor-executable program code to cause system 720 to acquire image data and generate images therefrom, and storage device 730 for storing the program code. Storage device 730 may comprise one or more fixed disks, solid-state random-access memory, and/or removable media (e.g., a thumb drive) mounted in a corresponding interface (e.g., a Universal Serial Bus port).

Storage device 730 stores program code of control program 731. One or more processing units 722 may execute control program 731 to control CT imaging elements of scanner 700 using CT system interface 724 and bed interface 725 to acquire CT data and to reconstruct CT images 733 therefrom. A CT image may be input to trained network 732 to generate a synthetic PET image as described above.

One or more processing units 722 may execute control program 731 to, in conjunction with PET system interface 723 and bed interface 725, control hardware elements to inject a radiopharmaceutical into a patient, move the patient into bore 712 past PET detectors of gantry 710, and detect photons emitted from the patient based on pulses generated by the PET detectors. The detected photons may be recorded in storage 730 as PET data 734, which may comprise raw (i.e., list-mode) data and/or sinograms.

Control program 731 may also be executed to reconstruct PET images 735 based on PET data 734 using any suitable reconstruction algorithm that is or becomes known. According to some embodiments, PET images 735 may be reconstructed based at least in part on CT images 733 (e.g., using a linear attenuation coefficient map determined from a CT image 733).

Control program 731 may be executed to compare a synthetic PET image to an intermediate PET image, to detect differences based on the comparison, to adapt PET data acquisition parameters based on the detected differences and on adaptation data 736, to acquire PET data based on the adapted PET data acquisition parameters, and to reconstruct a PET image from the thusly-acquired PET data.

PET images 735 and CT images 733 may be transmitted to terminal 740 via terminal interface 726. Terminal 740 may comprise a display device and an input device coupled to system 720. Terminal 740 may display the received PET images 735 and CT images 733. Terminal 740 may receive user input for controlling display of the data, operation of scanner 700, and/or the processing described herein. In some embodiments, terminal 740 is a separate computing device such as, but not limited to, a desktop computer, a laptop computer, a tablet computer, and a smartphone.

Each component of scanner 700 may include other elements which are necessary for the operation thereof, as well as additional elements for providing functions other than those described herein. Each functional component described herein may be implemented in computer hardware, in program code and/or in one or more computing systems executing such program code as is known in the art. Such a computing system may include one or more processing units which execute processor-executable program code stored in a memory system.

Those in the art will appreciate that various adaptations and modifications of the above-described embodiments can be configured without departing from the claims. Therefore, it is to be understood that the claims may be practiced other than as specifically described herein.

What is claimed is:

1. A molecular imaging scanner comprising:
a plurality of photon detectors; and
a processing unit to:
determine an anatomical image of an object;
input the anatomical image to a trained neural network to generate a synthetic functional image;
acquire molecular imaging data of the object based on acquisition parameters;
reconstruct a functional image based on the molecular imaging data; and
determine a difference between the functional image and the synthetic functional image.

2. A scanner according to claim 1, the processing unit to:
change one of the acquisition parameters based on the difference;
acquire second molecular imaging data of the object based on the changed acquisition parameters; and
reconstruct a second functional image based on the second molecular imaging data.

3. A molecular imaging scanner according to claim 2, wherein the second functional image is reconstructed based on the molecular imaging data and the second molecular imaging data.

4. A molecular imaging scanner according to claim 1, wherein reconstruction of the functional image and determination of the difference occur during acquisition of second molecular imaging data of the object based on the acquisition parameters.

5. A molecular imaging scanner according to claim 2, wherein the second functional image is reconstructed based on the molecular imaging data, the second molecular imaging data and the third molecular imaging data.

6. A molecular imaging scanner according to claim 2, wherein the difference is greater activity in a region of the functional image than in the region of the synthetic functional image, the one of the acquisition parameters is acceptance angle, and the change is a decrease in the acceptance angle with respect to the region.

7. A molecular imaging scanner according to claim 2, further comprising a table to support the object, wherein the difference is less activity in a region of the functional image than in the region of the synthetic functional image, the one of the acquisition parameters is a speed of the table, and the change is a decrease in the speed of the table.

8. A method comprising:
determining an anatomical image of an object;
inputting the anatomical image to a trained neural network to generate a synthetic functional image;
acquiring molecular imaging data of the object based on acquisition parameters;
reconstructing a functional image based on the molecular imaging data;
comparing the functional image and the synthetic functional image; and
determining whether to change one of the acquisition parameters based on the comparison.

9. A method according to claim 8, further comprising:
if it is determined to change one of the acquisition parameters based on the comparison:
changing the one of the acquisition parameters;
acquiring second molecular imaging data of the object based on the changed acquisition parameters; and
reconstructing a second functional image based on the second molecular imaging data; and
if it is not determined to change one of the acquisition parameters based on the comparison:
acquiring third molecular imaging data of the object based on the acquisition parameters; and
reconstructing a third functional image based on the molecular imaging data and the third molecular imaging data.

10. A method according to claim 9, wherein the second functional image is reconstructed based on the molecular imaging data and the second molecular imaging data.

11. A method according to claim 9, wherein reconstructing the functional image and determining whether to change one of the acquisition parameters occur during acquisition of fourth molecular imaging data of the object based on the acquisition parameters.

12. A method according to claim 11, wherein the second functional image is reconstructed based on the molecular imaging data, the second molecular imaging and the fourth molecular imaging data.

13. A method according to claim 11, wherein the third functional image is reconstructed based on the molecular imaging data and the third molecular imaging data and the fourth molecular imaging data.

14. A method according to claim 9, wherein the change is a decrease in acceptance angle.

15. A method according to claim 9, wherein the change is a decrease in table speed.

16. A non-transitory medium storing program code, the program code executable by at least one processing unit to cause a computing system to:
determine an anatomical image of an object;
input the anatomical image to a trained neural network to generate a synthetic functional image;
acquire molecular imaging data of the object based on acquisition parameters;
reconstruct a functional image based on the molecular imaging data; and
determine a difference between the functional image and the synthetic functional image.

17. A medium according to claim 16, the program code executable by at least one processing unit to cause a computing system to:
change one of the acquisition parameters based on the difference;
acquire second molecular imaging data of the object based on the changed acquisition parameters; and
reconstruct a second functional image based on the second molecular imaging data.

18. A medium according to claim 17, wherein the second functional image is reconstructed based on the molecular imaging data and the second molecular imaging data.

19. A medium according to claim 16, wherein reconstruction of the functional image and determination of the difference occur during acquisition of second molecular imaging data of the object based on the acquisition parameters.

20. A medium according to claim 17, wherein reconstruction of the functional image and determination of the difference occur during acquisition of second molecular imaging data of the object based on the acquisition parameters, and
the second functional image is reconstructed based on the molecular imaging data, the second molecular imaging data and the third molecular imaging data.
